(19) United States
(12) Patent Application Publication
(10) Pub. No.: US 2025/0001204 A1
(43) Pub. Date: Jan. 2, 2025

(54) LASER ILLUMINATION DEVICE FOR TREATING PERIPHERAL ARTERIAL OCCLUSIVE DISEASE

(71) Applicants: HOU-CHIEH LEE, Taoyuan City (TW); TING-TING LAM, Taoyuan City (TW)

(72) Inventors: HSUN-FU LEE, Taoyuan City (TW); HOU-CHIEH LEE, Taoyuan City (TW); CHIH-CHUNG WU, Taoyuan City (TW); TING-TING LAM, Taoyuan City (TW)

(73) Assignees: HOU-CHIEH LEE, Taoyuan City, TW (TW); TING-TING LAM, Taoyuan City, TW (TW)

(21) Appl. No.: 18/755,668

(22) Filed: Jun. 27, 2024

Related U.S. Application Data
(60) Provisional application No. 63/510,891, filed on Jun. 29, 2023.

(51) Int. Cl. A61N 5/067; A61N 5/06

(52) U.S. Cl. CPC A61N 5/067; A61N 2005/0642; A61N 2005/0652

(57) ABSTRACT

The present invention provides a laser illumination device for treating a peripheral artery occlusive disease, which comprises: a fixing frame, which includes a fixing member and a bracket, the fixing member is disposed on the top of the bracket, and the inner side of the fixing member is disposed with a plurality of a first laser diodes which are used to emit laser light with a wavelength between 620 and 700 nm, and a plurality of a second laser diodes which are used to emit laser light with a wavelength between 900 and 1100 nm; a power source, which is disposed on the fixing frame and is used to supply power to the plurality of laser diodes; and a tripod, which is disposed at the bottom of the bracket to support the fixing frame.

### CROSS-REFERENCE TO RELATED APPLICATIONS

This application claims priority to Taiwan Application No. 63/510,891 filed on Jun. 29, 2023, the entire content of which is incorporated herein by reference.

### FIELD OF THE INVENTION

The present invention provides afoot medical laser illumination device with two or more laser light sources, particularly a medical laser illumination device for treating a peripheral arterial occlusive disease.

### BACKGROUND OF THE INVENTION

A peripheral arterial occlusive disease has increased rapidly in recent years and is a heavy burden on public health. One of the main reasons is diabetes, and the most significant symptom is foot ulcer. The global average prevalence of foot disease among diabetic patients is 6.4%. Diabetic foot ulcers have a poor prognosis and are one of the main causes of disability and death in diabetic patients. The annual mortality rate of patients with diabetic foot ulcers is as high as 11%. Common conditions include infection, gangrene, cellulitis, etc. In severe cases, amputation is necessary. The mortality rate of amputation patients is as high as 22%. Most amputation patients lose economic productivity and mobility, and have to rely on others to take care of them, which is a heavy blow to the patient and his family.

One of the mechanisms that makes diabetic foot ulcers difficult to treat is that the phosphatidylserine receptors on macrophages are glycated, which blocks the recognition of apoptotic cells, leading to an increase in pro-inflammatory substances and making it difficult to subside inflammation. Another mechanism is the production of glycation end products and inflammatory mediators, which cause the apoptosis of fibroblasts and blood vessel-related cells, making it difficult to form a granulation tissue. From these two mechanisms, it can be seen that the best way to treat diabetic foot ulcers is to simultaneously reduce inflammation, relax blood vessels, and promote tissue and blood vessel proliferation to increase blood flow for effective treatment.

Previously, there have been patents utilizing multiple wavelengths of light sources for phototherapy on affected areas of different diseases. For example, WO2014/146029A1 uses light sources with wavelengths of 350˜480 nm and 590˜960 nm to relieve muscle and joint inflammation and pain, relieve acne or fungal infections, increase bone density, and promote plant growth.

U.S. Ser. No. 10/525,275B2 (and U.S. Ser. No. 11/524,173B2) uses dual-wavelength incoherent light sources (blue LEDs of 400˜420 nm and long-wavelength light sources of 620˜640 nm produced by irradiating fluorescent agents) to regulate nitric oxide (NO) in organisms. The function of shorter wavelength light is to increase intracellular NO production, and the function of longer wavelength light is to promote NO transport to the outside of cells and promote hair growth. Extracellular NO can promote vasoconstriction to increase blood flow.

JP2013146529A uses blue light, yellow light, and red light LED to promote collagen synthesis, inhibit bacterial growth, and inhibit melanin formation.

CN205339869U uses blue light (400˜490 nm), red light (600˜690 nm), and infrared light (780˜970 nm) laser or LED for the treatment of skin ulcers, burns, and trauma. However, because the treatment method is hand-held contact, it is easy to cause cross-infection.

The medical community has long confirmed the safety and efficacy of laser phototherapy for pain relief. However, there are few clinical reports regarding its use for diabetic foot ulcers. Although these clinical reports demonstrate that patients receiving phototherapy have better outcomes compared to those who do not, the current number of participants is still too small, and the dosages (light intensity and duration) and wavelengths are inconsistent. This makes comparisons difficult and standardization impossible. Therefore, sufficient evidence has yet to be accumulated to establish clinical guidelines. Currently, the application of laser phototherapy for diabetic foot ulcers is limited to medical research and has not been widely implemented. The speculated reasons for this are threefold: (1) Diabetic patients generally have poor health, and the presence of a foot ulcer indicates a severe condition. It is already challenging to slow the ulcer's progression, and reducing its size is even more difficult. Complete healing of the ulcer is rare. (2) In the past, laser light had low energy and small treatment areas, with a treatment beam diameter of less than 0.3 cm². To treat an affected area of about 10 cm², dozens of applications are required, which is time-consuming for both medical staff and patients, often leading to a loss of patience. (3) Previous lasers emitted a single wavelength (mostly red light with shorter wavelength), which only reached the surface of the skin and could not penetrate deep into the tissue, making it difficult to significantly increase blood perfusion.

According to previous research, in clinical practice, physicians must consider variables such as the patient's skin color, thickness, and the severity of the ulcer to determine the appropriate power density, energy density, and the number of irradiations within a treatment course. Therefore, it is necessary to adjust the power density and irradiation time accordingly.

### SUMMARY OF THE INVENTION

The present invention provides a laser illumination device for treating a peripheral artery occlusive disease, which comprises: a fixing frame, which includes a fixing member and a bracket, the fixing member is disposed on the top of the bracket, and the inner side of the fixing member is disposed with a plurality of a first kind of laser diodes which are used to emit laser light with a wavelength between 620 and 700 nm, and a plurality of a second kind of laser diodes which are used to emit laser light with a wavelength between 900 and 1100 nm; a power source, which is disposed on the fixing frame and is used to supply power to the plurality of the first kind and the second kind of laser diodes; and a tripod, which is disposed at the bottom of the bracket to support the fixing frame, wherein the plurality of laser diodes are used to emit lasers of different wavelengths to irradiate the affected area of a patient suffering from a peripheral arterial occlusive disease.

### BRIEF DESCRIPTION OF THE DRAWINGS

FIG. 1 is an example diagram of the laser illumination device of the present invention used to treat a peripheral arterial occlusive disease.

FIG. 2 is the structural design of the laser illumination device of the present invention.

FIG. 3 is a timing diagram of red light and near-infrared light alternatingly emitting light.

FIG. 4 shows an irradiation method in which pyramid-shaped laser light sources are appropriately spaced to obtain a uniform irradiation intensity.

FIG. 5 is a series of photographs taken during the treatment process of a subject using the laser illumination device of the present invention. FIGS. 5A to 5I are photographs taken on different dates during the treatment process.

### DETAILED DESCRIPTION OF THE INVENTION

The technical problem to be solved by the present invention is a multi-wavelength medical laser device for treating the peripheral arterial occlusive disease.

In order to solve the above problem, the present invention provides a laser device that irradiates the affected area with multiple wavelengths. The mechanisms of action and the penetration depths of different wavelengths vary. When different wavelengths act simultaneously, they produce a complementary synergistic effect, resulting in better therapeutic efficacy compared to a single wavelength.

Existing phototherapy devices include both red light and near-infrared lasers. In the past, the differences between the two were not well understood. However, recent advances in molecular biology have revealed that their mechanisms of action are significantly different. Red light acts on the cytochrome C oxidase (CCO) located in the mitochondrial membrane within the cell. Upon exposure to red light, there is an increase in the release of reactive oxygen species (ROS) and nitric oxide (NO), an enhancement in ATP synthesis, as well as an increased release of calcium ions (Ca²⁺), potassium ions (K⁺), and cyclic ANP (cAMP) from mitochondria into the cytoplasm. Near-infrared light, on the other hand, acts on ion channels in the mitochondrial membrane surrounded by water molecules adsorbed in its vicinity. The absorption spectrum of these water molecules differs significantly from that of free water molecules, absorbing near-infrared light around 1000 nm. This localized heating opens ion-selective channels, facilitating the release of large amounts of calcium ions (Ca²⁺) into the cytoplasm, triggering a cascade of reactions.

The key to the mechanism by which red light and infrared light increase blood flow lies in nitric oxide (NO) and calcium ions (Ca²⁺). Nitric oxide is a major neuronal signaling molecule with the ability to trigger vasodilation. Nitric oxide first stimulates soluble guanylate cyclase, leading to the formation of cyclic guanosine monophosphate (cGMP, an intracellular signaling molecule). Then, cGMP activates protein kinase G, leading to the reabsorption of calcium ions and opening of potassium channels activated by calcium ions. Due to the subsequent decrease in calcium ion concentration, myosin light-chain kinase is prevented from phosphorylating myosin molecules, causing the smooth muscle cells lining the blood vessels and lymphatic vessels to become relaxed, thereby increasing blood flow and lymph flow.

The increase in intracellular ATP and nitric oxide (NO) regulates inflammation-related transcription factors, thereby significantly reducing inflammation. These factors include nuclear factor-κB (NF-κB), prostaglandin E2, tumor necrosis factor-α (TNF-α), cyclooxygenase-2, and interleukin-1β (IL-1β).

The efficacy of phototherapy extends beyond vasodilation, increased blood flow, and anti-inflammatory effects. It also enhances the proliferation of fibroblasts, keratinocytes, endothelial cells, and lymphocytes. When mitochondria are stimulated by light, signaling pathways are initiated and transcription factors are upregulated, ultimately leading to an increase in growth factors. Phototherapy can enhance neovascularization, promote angiogenesis, and increase collagen synthesis, thereby improving the treatment of diabetic foot ulcers.

For chronic conditions such as diabetic foot ulcers, the treatment process involves first reducing inflammation, followed by re-epithelialization with new epithelial cells, and then remodeling of connective tissue to achieve complete healing. In recent years, it has been discovered that the canonical Wnt/β-catenin signal pathway plays a key role in this process, and this pathway is easily modulated by red light and near-infrared light. This pathway explains how red and near-infrared light assist in treating diabetic foot ulcers. However, existing methods have not yet achieved optimal effectiveness. The inventors believe that this is because existing methods fail to penetrate deeply into the skin. The present invention employs multiple wavelengths and large-area irradiation to achieve deeper penetration of light.

It is known that, in the human body, longer wavelengths generally penetrate deeper. Once light enters the human body, it is absorbed and scattered. The primary absorbers within the human body are melanin, hemoglobin, and water molecules. Comparatively, light with wavelengths between 600 and 1300 nm is less readily absorbed, creating an optical window in the human body that allows light to enter. Among these, 660 nm red light and 980 nm near-infrared laser light penetrate the human body more effectively through scattering. This is because these two wavelengths are less readily absorbed by melanin, hemoglobin (including both oxyhemoglobin (HbO₂) and deoxyhemoglobin (Hb), which have different absorption spectra), and water molecules. Approximately 95% of the hemoglobin in human arterial blood is oxyhemoglobin. After entering the microcirculation for oxygen-carbon dioxide exchange, only about 70% of the hemoglobin in the venous blood remains oxygenated. On average, most of the hemoglobin remains oxygenated. Oxyhemoglobin exhibits a low absorbance in the 650 to 700 nm range, allowing light within this wavelength to penetrate tissues more effectively. Considering the absorbance of subcutaneous fat, there is a low absorbance region around 980 nm, allowing 980 nm light to penetrate more easily through subcutaneous fat and affect the blood vessels beneath it. Due to the relatively low absorbance of epidermis, dermis, and subcutaneous tissue (rich in fat) for 660 nm red light and 980 nm near-infrared light, coupled with strong scattering properties, these wavelengths compared to others in the 600 to 1300 nm range are more prone to scattering and less absorption. Consequently, they can penetrate deeper into the body, leading to enhanced therapeutic efficacy. Considering the anatomy of the skin and hemodynamics, the primary resistance to blood flow is in the arterioles, not in the capillaries. Arterioles have three layers: the innermost layer is the tunica intima, which contains endothelial cells and the internal elastic lamina; the outermost layer is the tunica externa, which contains elastic fibers and collagen fibers; between the tunica intima and the tunica externa is the tunica media, which contains smooth muscle and elastic fibers and is the primary factor in regulating blood flow. Arterioles are more concentrated in the subcutaneous tissue and deeper layers, while only capillaries are present in the superficial dermis. If only 660 nm red light is used, it can only dilate the capillaries and not the deeper arterioles, resulting in overall limited efficacy. If 660 nm red light and 980 nm near-infrared light can be simultaneously irradiated on the same area, causing both superficial and deep blood vessels to dilate simultaneously, the effect will undoubtedly be more significant.

Furthermore, the present invention also uses multiple laser sources to form a large-area array to replace the existing small-area light sources, allowing the therapeutic light to penetrate deeper into the human body. A surgical laser scalpel relies on the coherence of a large number of photons to produce constructive interference, enabling it to cut through human tissue or coagulate blood, which requires high power. However, photons scattered by tissues inside the body lose their coherence and can no longer produce constructive interference, rendering them ineffective for cutting or coagulation. For lasers used in treating peripheral artery occlusive disease, the intensity does not need to be as powerful as that of ablative lasers like surgical laser scalpels. Additionally, although photons scattered by tissues lose their coherence, this does not impede their ability to stimulate mitochondria. As previously mentioned, human tissues exhibit lower absorption and higher scattering for 660 nm red light and 980 nm near-infrared light. After multiple scattering events, these wavelengths can penetrate deeper into body tissues, leading to the dilation of deeper blood vessels. Previous literature using computer simulations to account for the absorption levels in individuals with different skin tones has established the relationship between irradiation area (represented by beam diameter) and penetration depth. It has been found that the larger the irradiation area, the deeper the laser light penetrates.

Regarding the appropriate light intensity, previous literature has indicated that an irradiance of only 0.016 W/cm² is too low to be effective. Therefore, some literature suggests that a single irradiation's accumulated energy density (radiance exposure, the time-integral of irradiance) should be in the range of 3-7 J/cm² to be effective. Excessive power or prolonged exposure still raises concerns regarding efficacy and safety. Therefore, the international laser safety standard (IEC 60825-1:2014) stipulates that for non-ablative therapeutic lasers within the red to near-infrared range, the upper limit is 0.2 W/cm². To prevent human error, such as forgetting to turn off the power and resulting in excessive exposure time, it is best to have an automatic timer switch that shuts off the power.

The present invention provides a laser illumination device for treating peripheral artery occlusive disease, which comprises: a fixing frame, which includes a fixing member and a bracket, the fixing member is disposed on the top of the bracket, wherein the area of the inner side of the fixing member is greater than 25 cm², and the inner side of the fixing member is equipped with a plurality of first laser diodes which are used to emit laser light with a wavelength between 620 and 700 nm, and a plurality of second laser diodes which are used to emit laser light with a wavelength between 900 and 1100 nm; a power source, which is disposed on the fixing frame and is used to supply power to the plurality of laser diodes; and a tripod, which is disposed at the bottom of the bracket to support the fixing frame, wherein the plurality of laser diodes are used to emit lasers of different wavelengths to irradiate the affected area of a patient suffering from peripheral arterial occlusive disease.

In the present invention, the area of the plurality of laser diodes disposed inside the fixing member is greater than 25 cm². In an embodiment, the area of the plurality of laser diodes disposed inside the fixing member ranges from 25 to 800 cm². In a preferred embodiment, the area of the plurality of laser diodes disposed inside the fixing member ranges from 25 to 600 cm². In a more preferred embodiment, the area of the plurality of laser diodes disposed inside the fixing member ranges from 25 to 400 cm².

In another embodiment, the material of the fixing member is aluminum, copper, or aluminum nitride.

In an embodiment, the laser illumination device further comprises a heat dissipation member disposed outside the fixing member to dissipate the heat generated by the plurality of laser diodes.

In another embodiment, the number of each of the plurality of the first kind of laser diodes and the plurality of the second kind of laser diodes is at least 8. In a preferred embodiment, the number of each of the plurality of the first kind of laser diodes and the plurality of the second kind of laser diodes is at least 15. In a more preferred embodiment, the number of each of the plurality of the first kind of laser diodes and the plurality of the second kind of laser diodes is at least 20.

In an embodiment, the distance between the plurality of laser diodes on the inner side of the fixing member and the affected area of the patient is greater than 5 cm. In a preferred embodiment, the distance between the plurality of laser diodes on the inner side of the fixing member and the affected area of the patient is greater than 7 cm. In a more preferred embodiment, the distance between the plurality of laser diodes on the inner side of the fixing member and the affected area of the patient is greater than 10 cm.

In another embodiment, the inner side of the fixing member has a planar or curved shape.

In an embodiment, the plurality of first laser diodes and the plurality of second laser diodes are installed in an interleaved manner on the inner side of the fixing member, ensuring that the maximum and minimum light intensities on the irradiated surface are within ±30% of the average light intensity. In a preferred embodiment, the plurality of first laser diodes and the plurality of second laser diode are installed in an interleaved manner on the inner side of the fixing member, ensuring that the maximum and minimum light intensities on the irradiated surface are within ±40% of the average light intensity. In a more preferred embodiment, the plurality of first laser diodes and the plurality of second laser diode are installed in an interleaved manner on the inner side of the fixing member, ensuring that the maximum and minimum light intensities on the irradiated surface are within ±50% of the average light intensity.

In another embodiment, the power source is further connected to a timing device so that the power source automatically turns off at a set time.

In an embodiment, the laser illumination device further comprises a programmable digital controller for controlling the power source, ensuring that the optical power density of the laser light emitted by the plurality of laser diodes on the patient's affected area ranges from 8 to 200 mW/cm². In a preferred embodiment, the optical power density ranges from 10 to 100 mW/cm². In a more preferred embodiment, the optical power density ranges from 20 to 50 mW/cm². In the present invention, the power source is connected to the plurality of laser diodes. The programmable digital controller can control the power of the plurality of laser diodes.

In another embodiment, the power source provides power in an alternating manner, with an alternating frequency range of 40 to 1000 Hz. In a preferred embodiment, the power source provides power in an alternating manner, with an alternating frequency range of 60 to 800 Hz. In a more preferred embodiment, the power source provides power in an alternating manner, with an alternating frequency range of 100 to 500 Hz.

In an embodiment, an infrared temperature sensor is installed on the fixing frame for sensing the temperature of the irradiated human skin.

The present invention provides a method for treating the peripheral artery occlusive disease comprising: using the above-mentioned laser illumination device to emit multiple laser lights to irradiate the affected area of an individual suffering from peripheral artery occlusive disease, wherein the laser lights comprise a first laser light and a second laser light, the first laser light has a wavelength of 620-700 nm, and the second laser light has a wavelength of 900˜1100 nm, and the optical power density of the first laser light and the second laser light irradiated on the affected area of the individual ranges from 8 to 200 mW/cm².

In an embodiment, the peripheral arterial occlusive disease comprises diabetes.

In another embodiment, the affected area is a damaged area caused by peripheral artery occlusion. In a preferred embodiment, the affected area is a foot ulcer caused by diabetes.

In an embodiment, the irradiation distance between the laser light and the individual is more than 5 cm. In a preferred embodiment, the irradiation distance between the laser light and the individual is more than 7 cm. In a more preferred embodiment, the irradiation distance between the laser light and the individual is more than 10 cm.

In another embodiment, the optical power of the plurality of laser lights ranges from 10 to 100 mW/cm². In a preferred embodiment, the optical power of the plurality of laser lights ranges from 20 to 50 mW/cm².

In an embodiment, the first laser light and the second laser light emit alternately at a frequency ranging from 40 to 1000 Hz. In a preferred embodiment, the first laser light and the second laser light emit alternately at a frequency ranging from 60 to 800 Hz. In a preferred embodiment, the first laser light and the second laser light emit alternately at a frequency ranging from 100 to 500 Hz.

In another embodiment, the irradiation time of the plurality of laser lights is 10-100 minutes. In a preferred embodiment, the irradiation time of the plurality of laser lights is 20-50 minutes. In a more preferred embodiment, the irradiation time of the plurality of laser lights is 30-40 minutes.

In an embodiment, the plurality of laser lights are irradiated at least once a week. In a preferred embodiment, the plurality of laser lights are irradiated at least twice a week.

In an embodiment, the plurality of laser lights are irradiated for at least one week. In a preferred embodiment, the plurality of laser lights are irradiated for at least two weeks. In a more preferred embodiment, the plurality of laser lights are irradiated for at least one month.

### Examples

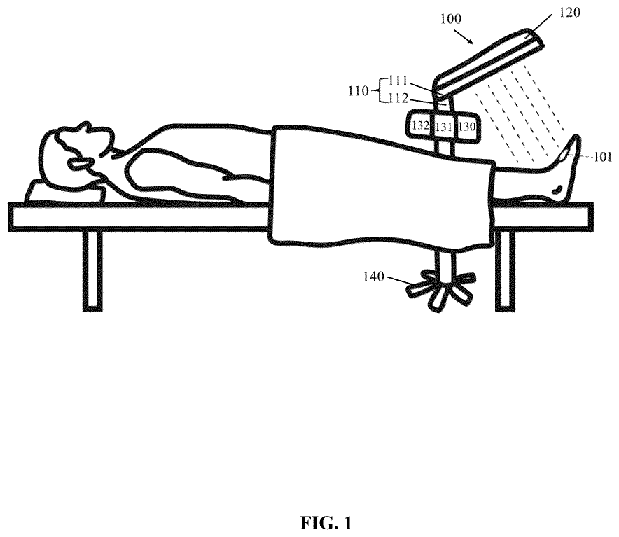

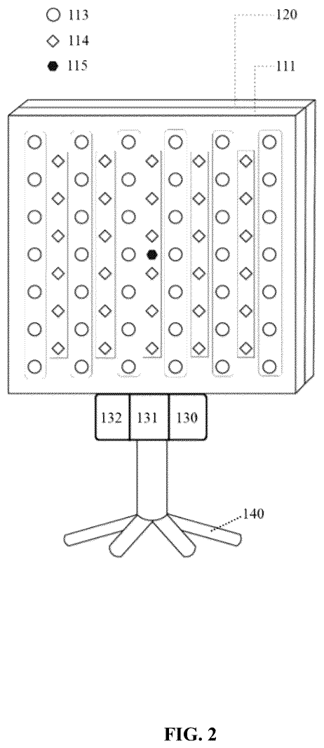

FIG. 1 is an example diagram of the laser illumination device of the present invention used for treating a peripheral artery occlusive disease. The laser illumination device 100 comprises a fixing frame 110, which includes a fixing member 111 and a bracket 112, and the fixing member 111 is disposed on the top of the bracket 112, wherein the inner side of the fixing member 111 is disposed with a plurality of laser diodes 113, 114 (as shown in FIG. 2) which are used to emit laser light of different wavelengths to irradiate the affected area 101 of a patient suffering from the peripheral arterial occlusive disease; a heat dissipation member 120 disposed outside the fixing member 111 to dissipate the heat generated by the plurality of laser diodes; a power source 130, which is disposed on the bracket 112 and is used to supply power to the plurality of laser diodes; and a tripod 140, which is disposed at the bottom of the bracket 112 to support the fixing frame.

In addition, the power source 130 is further connected to a timing device 131. The function of the timing device 131 is to allow the power source 130 to automatically turn off at a set time.

The power source 130 is also connected to a programmable digital controller 132. The function of the programmable digital controller 132 is to control the power source 130, thereby adjusting the power of the light emitted by the plurality of laser diodes.

FIG. 2 is the structural design of the laser illumination device of the present invention. The area of the inner side of the fixing member 111 of the laser illumination device is greater than 25 cm² to carry the plurality of laser diodes. In addition, the plurality of laser diodes are divided into a first kind of laser diodes 113 and a second kind of laser diodes 114, wherein the first kind of laser diodes 113 can emit the laser light with a wavelength between 620 and 700 nm, and the second kind of laser diodes 114 can emit the laser light with a wavelength between 900 and 1100 nm. Therefore, a heat dissipation member 120 is disposed on the outside of the fixing member 111 to dissipate the heat generated by the plurality of laser diodes.

In addition, an infrared temperature sensor 115 is further provided inside the fixing member 111 for sensing the skin temperature of a patient.

The plurality of laser diodes are connected to a power source 130, and the power source 130 is used to supply power to the plurality of laser diodes. The power source 130 is further connected to a timing device 131, and the function of the timing device 131 is to allow the power source 130 to automatically turn off at a set time.

In addition, the power source 130 is also connected to a programmable digital controller 132. The function of the programmable digital controller 132 is to control the power source 130, thereby adjusting the power of the light emitted by the plurality of laser diodes. In addition, the function of the tripod 140 is to support the fixing frame.

### Example 1: Laser Sources of Different Wavelengths Emit Light Alternatingly to Facilitate Heat Dissipation

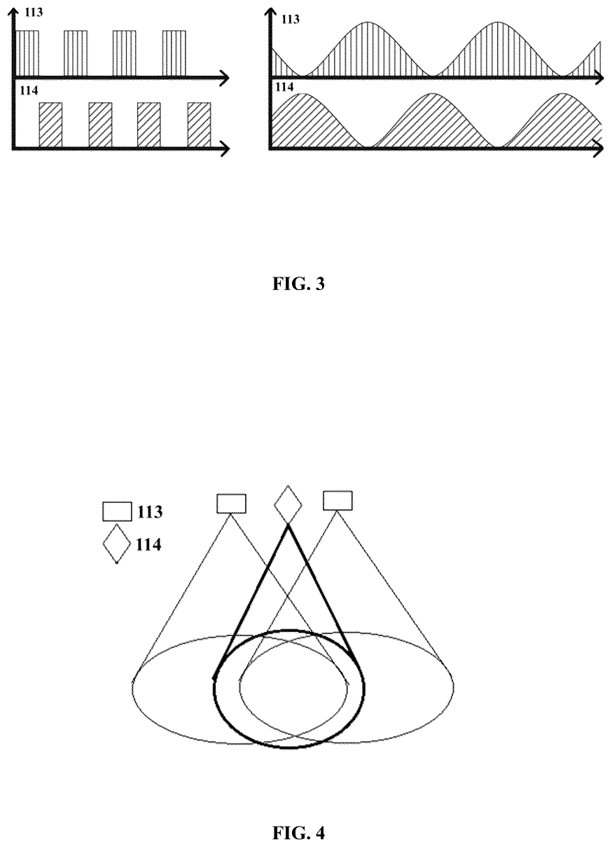

In current technology, heat dissipation is the primary bottleneck for the light power of laser diodes. The maximum power of the laser is constrained by its capability to dissipate heat. If excessive heat cannot be eliminated, the laser diode will be subject to irreversible damage. The present invention allows laser sources of different wavelengths (i.e., red light and near-infrared light) to emit light alternatingly, effectively enabling the laser diodes to dissipate heat and reduce the probability of damage. As shown in FIG. 3, the luminous waveforms of the first kind of laser diodes (red light) 113 and the second kind of laser diodes (near-infrared light) 114 can be pulse waves (left side of FIG. 3) or sine waves (right side of FIG. 3), glowing alternatingly with each other.

Considering the persistence of vision in the human eye, the alternating emission should ideally exceed 40 Hz to avoid causing discomfort from visible flicker. Considering also the structure of the laser diode, the heating occurs within the resonant cavity of the diode, which has approximately tens of micrometers (μm) in length, width, and height. For such a small volume, if there is one hundredth of a second interval to allow it to dissipate heat, significant effects can be achieved. If the interval is as short as one-thousandth of a second, it will not be sufficient for dissipating heat. Therefore, the alternating frequency is preferably faster than 40 Hz, but no more than 1 K Hz. Furthermore, in order to have better heat dissipation capability, the fixing member for installing the laser sources should be made of materials with good thermal conductivity, such as aluminum, copper, or aluminum nitride. The fixing frame can also be connected to other heat dissipation devices, such as a thermoelectric cooler (TE cooler), and can be further connected to heat dissipation fins and fans to facilitate heat dissipation.

### Example 2: A Non-Contact Infrared Sensor is Installed Between the Laser Source Arrays to Sense Human Body Surface Temperature

It is known that the thermal effects produced by 660 nm and 980 nm wavelengths in a human tissue are not significant. For a healthy individual, even at the IEC 60825-1 specified upper limit of 200 mW/cm², there is no risk of damage or discomfort due to overheating. However, for a patient with the peripheral arterial occlusive disease who has poor peripheral blood circulation and nearly disabled nerves, the possibility of overheating and injury due to phototherapy cannot be completely excluded. Therefore, a preferred preventive implementation is to install a non-contact infrared sensor (not shown in the figure), such as the TP006 produced by Texas Instruments, among the laser source array as illustrated in FIG. 2. Because the TMP006 has high sensitivity to infrared wavelengths in the range of 4-16 μm and almost no response to 660 nm and 980 nm, it will not be affected by interference from the device's own lasers. Moreover, the TMP006's internal thermopile receives infrared radiation in a conical range of approximately 90 degrees, making it less susceptible to interference from adjacent laser sources. It only receives infrared radiation from the human body directly in front of it, allowing for accurate and non-contact temperature measurement of the human body.

### Example 3: The Laser Sources are Arranged in an Interlaced Pattern to Form an Illumination Surface with Uniform Intensity

42 red light laser diodes form 7 red light modules, and 30 infrared light laser diodes form 5 infrared light modules, as shown in FIG. 2. The laser light emitted by the laser diodes is not collimated light but a Gaussian beam that diffuses in a cone shape. A single laser diode has a horizontal diffusion angle of about 10 degrees and a vertical diffusion angle of about 20 degrees. The single laser diode, based on its divergence angle, will form an elliptical illumination area on the target surface at a distance of 20 cm from the chip, with a long axis of 7.05 cm and a short axis of 3.5 cm. As shown in FIG. 4, two first type laser sources 113 are installed with a 3.52 cm interval, with a second type laser source 114 placed in between. This arrangement allows the Gaussian beams from each laser source to complement each other in intensity, thereby forming a continuous illuminated area on the target surface where the maximum and minimum luminosities are within ±30% of the average luminosity. Both types of laser sources will irradiate the same area. To complement each other's intensity to form an irradiation surface with uniform luminosity, a preferred implementation is that the number of each type of laser diodes is greater than 8. This arrangement provides a sufficiently large irradiation area, allowing the therapeutic light to penetrate deeper into the body, thereby enhancing the treatment effect. If a larger irradiation area is required, a handheld operation is impractical. A preferred implementation is to use a tripod to stand on the ground to support the fixing frame for installing the laser sources. The operator does not need to hold it, and the fixing frame can be kept at a distance from the patient to avoid cross-infection.

### Implementation Results 1

In the present invention, skin perfusion pressure (SPP) is used to test whether the previously obstructed peripheral arterial blood flow increases after receiving phototherapy. SPP measurement has been proven to help evaluate critical limb ischemia (CLI) in the peripheral arterial occlusive disease and is also the best indicator for determining amputation. The SPP of a healthy individual is greater than 50 mmHg, and if it is less than 30 mmHg, amputation is very likely necessary. The present invention employed the VMS-LDF1 laser Doppler skin perfusion pressure monitor (Moor Instruments, UK) for testing. In Cases 1 and 2, the subjects were severe diabetic patients admitted to the plastic surgery ward of a medical center in northern Taiwan. Their foot ulcers underwent two weeks of dual-wavelength (660 nm and 980 nm) phototherapy, totaling five sessions, each lasting 30 minutes. The average optical power was 40 mW/cm², and the laser array area was about 600 cm².
Case 1: The SPP was 41.4 mmHg before treatment and 58.6 mmHg after completing the treatment.
Case 2: The SPP was 34.9 mmHg before treatment and 48.4 mmHg after completing the treatment.

### Implementation Results 2

The present invention also used Image J software, developed by the National Institutes of Health (NIH), to calculate the area of the foot ulcer. The subjects were severe diabetic patients treated in the Department of Plastic Surgery at a medical center in southern Taiwan. Case 3 was an inpatient, and case 4 was an outpatient. The information on the dates before and after treatment, as well as the affected area (cm²) at that time, is shown in Table 1 and Table 2.

TABLE 1
Treatment date and affected area of case 3 (inpatient)

| | Debridement | | | | | | |
|---|---|---|---|---|---|---|---|
| Before | and | | | | | | |
| treatment | phototherapy | Phototherapy | Phototherapy | Phototherapy | Phototherapy | Phototherapy | Phototherapy |
| D | D + 1 | D + 4 | D + 5 | D + 7 | D + 8 | D + 11 | D + 12 |
| 7.0 | 7.2 | 7.0 | 6.4 | 5.7 | 4.9 | 4.1 | 3.5 |

TABLE 2
Treatment date and affected area of case 4 (outpatient)

| Before | Debridement and | | | |
|---|---|---|---|---|
| treatment | phototherapy | Phototherapy | Phototherapy | Phototherapy |
| D | D + 7 | D + 15 | D + 18 | D + 21 |
| 3.02 | 3.50 | 3.08 | 1.39 | 0.96 |

### Implementation Results 3

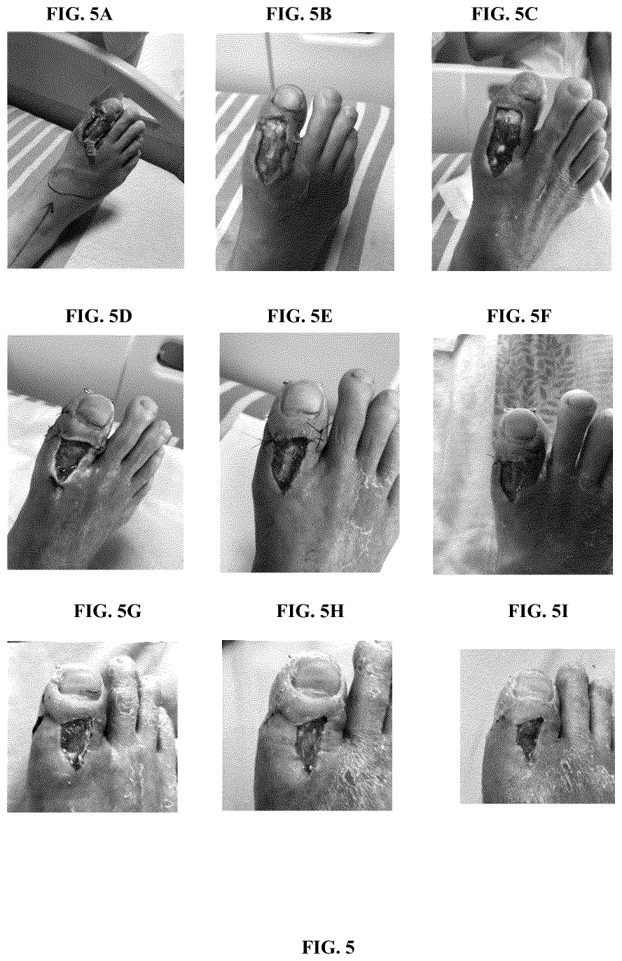

The present invention also used photos to record the affected areas receiving phototherapy. The subject was a diabetic patient admitted to the plastic surgery department of a medical center in northern Taiwan who suffered from ulcers due to trauma. Each phototherapy session was conducted as described in Implementation Result 1. FIG. 5 is a series of photographs taken during the treatment process of the subject using the laser illumination device. FIGS. 5A to 5I are photographs taken on different dates during the treatment process. The relevant dates and descriptions are shown in Table 3.

TABLE 3
Dates and descriptions of photographs taken during
the laser phototherapy process for the subject.

| Number | FIG. 5A | FIG. 5B | FIG. 5C | FIG. 5D |
|---|---|---|---|---|
| Date and | Admitted to | D + 3 | D + 8 | D + 14 Surgery |
| description | hospital | | | |
| | (D) | | | |

| FIG. 5E | FIG. 5F | FIG. 5G | FIG. 5H | FIG. 5I |
|---|---|---|---|---|
| D + 29 | D + 31 | D + 35 | D + 44 | D + 46 |
| | Discharged | | | |
| | from hospital | | | |

What is claimed is:

1. A laser illumination device for treating a peripheral artery occlusive disease, which comprises:
a fixing frame, which includes a fixing member and a bracket, the fixing member being disposed on a top of the bracket, wherein an area of an inner side of the fixing member is greater than 25 cm², and the inner side of the fixing member is disposed with a plurality of a first kind of laser diodes which are used to emit laser light with a wavelength between 620 and 700 nm, and a plurality of a second kind of laser diodes which are used to emit laser light with a wavelength between 900 and 1100 nm;
a power source, which is disposed on the fixing frame and is used to supply power to the plurality of the first kind and the second kind of laser diodes; and
a tripod, which is disposed at a bottom of the bracket to support the fixing frame, wherein the plurality of the first kind of laser diodes and the plurality of the second kind of laser diodes are used to emit laser light of different wavelengths to irradiate an affected area of a patient suffering from the peripheral arterial occlusive disease.

2. The laser illumination device of claim 1, which further comprises a heat dissipation member disposed outside the fixing member to dissipate heat generated by the plurality of laser diodes.

3. The laser illumination device of claim 1, wherein the number of each of the plurality of the first kind of laser diodes and the plurality of the second kind of laser diodes is at least 8.

4. The laser illumination device of claim 1, wherein a distance between the plurality of the first kind and/or the second kind of laser diodes on the inner side of the fixing member and the affected area of the patient is greater than 5 cm.

5. The laser illumination device of claim 1, wherein the inner side of the fixing member is in a planar or curved shape.

6. The laser illumination device of claim 1, wherein the plurality of the first laser diodes and the plurality of the second laser diode are respectively installed on the inner side of the fixing member in an interleaved manner, ensuring that a maximum and a minimum light intensities on an irradiated surface are within ±30% of an average light intensity.

7. The laser illumination device of claim 1, wherein the power source is further connected to a timing device so that the power source automatically turns off at a set time.

8. The laser illumination device of claim 1, which further comprises a programmable digital controller for controlling the power source, ensuring that a light power density of the laser light emitted by the plurality of the first kind and the second kind of laser diodes on the patient's affected area ranges from 8 to 200 mW/cm².

9. The laser illumination device of claim 1, wherein the power source provides the power in an alternating manner, with an alternating frequency range of 40 to 1000 Hz.

10. The laser illumination device of claim 1, which further comprises an infrared temperature sensor installed on the fixing member for sensing a skin temperature of the patient.
